(19) United States
(12) Patent Application Publication
(10) Pub. No.: US 2024/0096505 A1
(43) Pub. Date: Mar. 21, 2024

(54) INFORMATION PROCESSING APPARATUS, INFORMATION PROCESSING METHOD AND RECORDING MEDIUM

(71) Applicant: NEC Corporation, Minato-ku, Tokyo (JP)

(72) Inventors: Kazufumi Ikeda, Tokyo (JP); Mamoru Takeuchi, Tokyo (JP)

(73) Assignee: NEC Corporation, Minato-ku, Tokyo (JP)

(21) Appl. No.: 17/639,686

(22) PCT Filed: Mar. 24, 2021
(86) PCT No.: PCT/JP2021/012249

§ 371 Date: Sep. 29, 2022

(51) Int. Cl. G16H 70/60

(52) U.S. Cl. CPC G16H 70/60

(57) ABSTRACT

An information processing apparatus (2) includes: an obtaining unit (211) that obtains an infection information relating to an infected person that catches an infection disease; and an identifying unit (213) that identifies, as a contact person that possibly contacts the infected person, at least one target person staying in a stay area in which the infected person stays in at least a part of a time period during which the infected person stays in the stay area from a plurality of target persons based on the infection information and a history information (221) that is generated based on a plurality of target person images (IMG) that indicate the plurality of target persons, respectively.

### TECHNICAL FIELD

The present disclosure relates to a technical field of an information processing apparatus, an information processing method and a recording medium that are configured to identify a contact person that possibly contacts an infected person catching an infection disease, for example.

### BACKGROUND ART

A Patent Literature 1 discloses one example of an information processing apparatus that is configured to identify a contact person that possibly contacts an infected person catching an infection disease. Specifically, the Patent Literature 1 discloses a behavior management system that includes: a mobile terminal that receives an information from another mobile terminal by using a Near Field Communication such as a Bluetooth (a registered trademark) and generates, based on the received information, a behavior check information including a terminal unique information of another mobile terminal, a distance to another mobile terminal, a direction of another mobile terminal and a current time; and a server that determines a positional relationship between users based on the behavior check information. Furthermore, the Patent Literature 1 discloses that the behavior management system can be used to detect a closed contact person to the infected person catching the infection disease such as a COVID-19.

Additionally, there are Patent Literatures 2 to 4 as a background art document relating to the present disclosure.

### CITATION LIST

### Patent Literature

Patent Literature 1: JP2020-201287A
Patent Literature 2: JP2019-083395A
Patent Literature 3: JP2012-198717A
Patent Literature 4: JP2009-259269A

### SUMMARY

### Technical Problem

It is an example object of the present disclosure to provide an information processing apparatus, an information processing method and a recording medium that aims to an improvement of a technique disclosed in the background art document.

### Solution to Problem

One example aspect of an information processing apparatus includes: an obtaining unit that obtains an infection information relating to an infected person that catches an infection disease; and an identifying unit that identifies, as a first contact person that possibly contacts the infected person, at least one target person staying in a stay area in which the infected person stays in at least a part of a time period during which the infected person stays in the stay area from a plurality of target persons based on the infection information and a history information that is generated based on a plurality of target person images that indicate the plurality of target persons, respectively.

One example aspect of an information processing method includes: obtaining an infection information relating to an infected person that catches an infection disease; and identifying, as a first contact person that possibly contacts the infected person, at least one target person staying in a stay area in which the infected person stays in at least a part of a time period during which the infected person stays in the stay area from a plurality of target persons based on the infection information and a history information that is generated based on a plurality of target person images that indicate the plurality of target persons, respectively.

One example aspect of a recording medium is a recording medium on which a computer program that allows a computer to execute an information processing method is recorded, the information processing method includes: obtaining an infection information relating to an infected person that catches an infection disease; and identifying, as a first contact person that possibly contacts the infected person, at least one target person staying in a stay area in which the infected person stays in at least a part of a time period during which the infected person stays in the stay area from a plurality of target persons based on the infection information and a history information that is generated based on a plurality of target person images that indicate the plurality of target persons, respectively.

### BRIEF DESCRIPTION OF DRAWINGS

FIG. 1 is a block diagram that illustrates an entire configuration of an information processing system in a first example embodiment.

FIG. 2 is a block diagram that illustrates a configuration of an information processing apparatus in the first example embodiment.

FIG. 3 illustrates one example of a data structure of an authentication history DB.

FIG. 4 is a flow chart that illustrates a flow of a contact person identification operation that is performed by the information processing apparatus in the first example embodiment.

FIG. 5 conceptually illustrates one example of an operation for identifying a contact person on the authentication history DB.

FIG. 6 conceptually illustrates one example of the operation for identifying the contact person on the authentication history DB.

FIG. 7 conceptually illustrates one example of the operation for identifying the contact person on the authentication history DB.

FIG. 8 conceptually illustrates one example of the operation for identifying the contact person on the authentication history DB.

FIG. 9 is a block diagram that illustrates a modified example of the information processing apparatus in the first example embodiment.

FIG. 10 is a block diagram that illustrates an entire configuration of an information processing system in a second example embodiment.

FIG. 11 is a block diagram that illustrates a configuration of an information processing apparatus in the second example embodiment.

FIG. 12 illustrates one example of a data structure of a body temperature history DB.

FIG. 13 is a flow chart that illustrates a flow of an entry management operation that is performed by the information processing apparatus in the second example embodiment.

FIG. 14 is a block diagram that illustrates a configuration of an information processing apparatus in a third example embodiment.

FIG. 15 illustrates a thermal image in which a target person wearing a mask is included.

FIG. 16 is a block diagram that illustrates a configuration of an information processing apparatus in a fourth example embodiment.

### DESCRIPTION OF EXAMPLE EMBODIMENTS

Next, an example embodiment of an information processing apparatus, an information processing method and a recording medium will be described. In the below described description, the example embodiment of the information processing apparatus, the information processing method and the recording medium will be described by using an information processing system SYS.

### (1) Information Processing System SYS in First Example Embodiment

Firstly, the information processing system SYS in a first example embodiment will be described. Incidentally, in the below described description, the information processing system SYS in the first example embodiment is referred to as the “information processing system SYSa”. The information processing system SYSa in the first example embodiment is a system that is configured to authenticate target persons and identify a contact person that possibly contacts an infected person catching an infection disease from the authenticated target persons.

Here, a behavior management system disclosed in the above described Patent Literature 1 determines a positional relationship between a plurality of users having a plurality of mobile terminals, respectively, by using a Near Field Communication such as a Bluetooth (a registered trademark) to identify a contact person that possibly contacts an infected person. However, the behavior management system disclosed in the Patent Literature 1 identifies only the contact person that is so close to the infected person that an information can be receivable from the mobile terminal of the infected person and/or an information can be transmittable to the mobile terminal of the infected person, because it uses the Near Field Communication.

On the other hand, there is a possibility that a person catches an infection disease by means of the person other than the infected person contacting an object to which an airborne droplet of the infected person is adhered. Thus, it is preferable to also identify, as the contact person that possibly contacts the infected person (more specifically, that is possibly affected by the infection disease which the infected person catches), a person that is not so close to the infected person that an information can be receivable from the mobile terminal of the infected person and/or an information can be transmittable to the mobile terminal of the infected person but possibly contacts the object to which the airborne droplet of the infected person is adhered. However, the behavior management system disclosed in the Patent Literature 1 has a technical problem that it is not capable of identifying, as the contact person, the person that possibly contacts the object to which the airborne droplet of the infected person is adhered. Namely, the behavior management system disclosed in the Patent Literature 1 has a technical problem that there is possibly an omission of the contact person. Thus, the behavior management system disclosed in the Patent Literature 1 has a room for improvement.

Thus, next, the information processing system SYSa that is configured to solve the technical problem of the behavior management system disclosed in the Patent Literature 1 will be described.

### (1-1) Configuration of Information Processing System SYSa

(1-1-1) Entire Configuration of Information Processing System SYSa

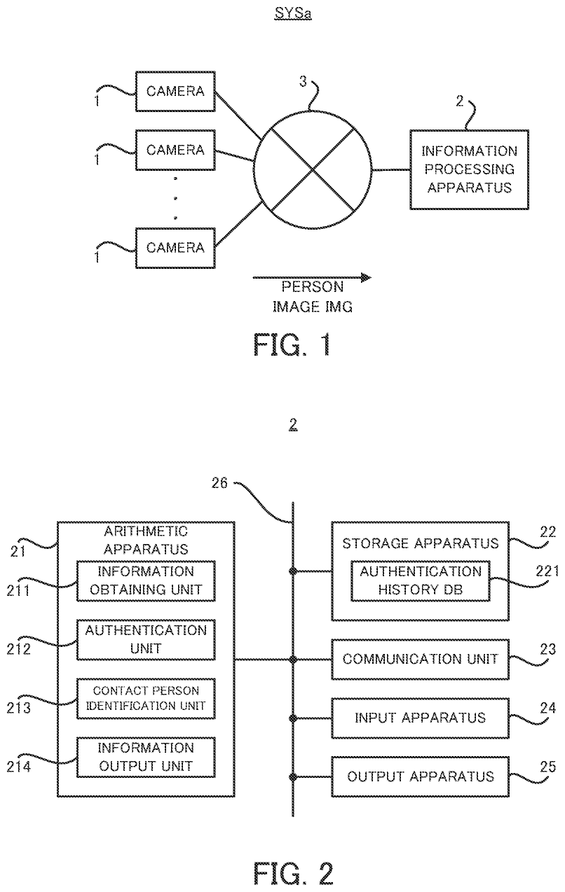

Firstly, with reference to FIG. 1, an entire configuration of the information processing system SYSa in the first example embodiment will be described. FIG. 1 is a block diagram that illustrates the entire configuration of the information processing system SYSa in the first example embodiment.

As illustrated in FIG. 1, the information processing system SYSa includes cameras 1 and an information processing apparatus 2. The information processing system SYSa includes the plurality of cameras 1, however, may include a single camera 1. The cameras 1 and the information processing apparatus 2 may be configured to communicate with each other through a communication network 3. The communication network 3 may include a wired communication network. The communication network 3 may include a wireless communication network.

The camera 1 is an imaging apparatus that is configured to capture an image of a target person (namely, a person) that is located in an imaging target range of the camera 1. Incidentally, in the below described description, the target person the image of which is captured by the camera 1 may be referred to as a captured person. The camera 1 generates a person image IMG that includes the target person the image of which is captured by the camera 1 by capturing the image of the target person. The person image IMG that indicates the target person may be typically an image in which the target person is included. Note that the “person image IMG in which the target person is included” may include an image that is generated by means of the camera 1 capturing the image of the target person that does not have an intention of wanting the camera 1 to capture the image of the target person. The “person image IMG in which the target person is included” may include an image that is generated by means of the camera 1 capturing the image of the target person that has the intention of wanting the camera 1 to capture the image of the target person. The camera 1 outputs the generated person image IMG to the information processing apparatus 2. Specifically, the camera 1 transmits the generated person image IMG to the information processing apparatus 2 through the communication network 3.

In the first example embodiment, the camera 1 is placed at a stay area in which the target person can stay. Typically, the plurality of cameras 1 may be placed at a plurality of different stay areas, respectively. Namely, each camera 1 may be placed at one stay area that corresponds to each camera 1. In this case, the camera 1 may capture the image of the target person that newly enters the stay area from an area that is different from the stay area. The camera 1 may capture the image of the target person that leaves the stay area to the area that is different from the stay area. The camera 1 may capture the image of the target person that is staying in the stay area.

A specific facility (namely, an area inside the facility) is one example of the stay area. At least one of a condominium, a hotel, an office building, a restaurant, a retail shop, a school and a plant is one example of the specific facility. A specific area in the facility is one example of the stay area. At least one of each room in the condominium, each room in the hotel, each room in the office building, each selling space in the retail shop, each classroom in the school and each block in the plant is one example of the specific area in the facility. A specific site is one example of the stay area. At least one of a site of the condominium, a site of the hotel, a site of the office building, a site of the retail shop, a site of the school and a site of the plant is one specific example of the specific site.

The information processing apparatus 2 receives the person image IMG that is transmitted from the camera 1 through the communication network 3. The information processing apparatus 2 performs a contact person identification operation for identifying the contact person by using the received person image IMG.

(1-1-2) Configuration of Information Processing Apparatus 2

Next, with reference to FIG. 2, a configuration of the information processing apparatus 2 in the first example embodiment will be described. FIG. 2 is a block diagram that illustrates the configuration of the information processing apparatus 2 in the first example embodiment.

As illustrated in FIG. 2, the information processing apparatus 2 includes an arithmetic apparatus 21, a storage apparatus 22 and a communication apparatus 23. Furthermore, the information processing apparatus 2 may include an input apparatus 24 and an output apparatus 25. However, the information processing apparatus 2 may not include at least one of the input apparatus 24 and the output apparatus 25. The arithmetic apparatus 21, the storage apparatus 22, the communication apparatus 23, the input apparatus 24 and the output apparatus 25 may be interconnected through a data bus 26.

The arithmetic apparatus 21 includes at least one of a CPU (Central Processing Unit), a GPU (Graphic Processing Unit) and a FPGA (Field Programmable Gate Array), for example. The arithmetic apparatus 21 reads a computer program. For example, the arithmetic apparatus 21 may read a computer program that is stored in the storage apparatus 22. For example, the arithmetic apparatus 21 may read a computer program that is stored in a non-transitory computer-readable recording medium by using a non-illustrated recording medium reading apparatus of the information processing apparatus 2. The arithmetic apparatus 21 may obtain (namely, download or read) a computer program from a non-illustrated apparatus that is placed outside the information processing apparatus 2 through the communication apparatus 23 (alternatively, other communication apparatus) The arithmetic apparatus 21 executes the read computer program. As a result, a logical functional block for performing an operation (for example, the above described contact person identification operation) that should be performed by the information processing apparatus 2 is implemented in the arithmetic apparatus 21. Namely, the arithmetic apparatus 21 is configured to serve as a controller for implementing the logical functional block for performing the operation (in other words, a processing) that should be performed by the information processing apparatus 2.

FIG. 2 illustrates one example of the logical functional block that is implemented in the arithmetic apparatus 21 for performing the contact person identification operation. As illustrated in FIG. 2, in the arithmetic apparatus 21, an information obtaining unit 211 that is one specific example of “an obtaining unit”, an authentication unit 212, a contact person identification unit 213 that is one specific example of “an identifying unit” and an information output unit 214 are implemented. Note that a detail of an operation of each of the information obtaining unit 211, the authentication unit 212, the contact person identification unit 213 and an information output unit 214 will be described later in detail, however, a summary thereof will be described briefly here. The information obtaining unit 211 obtains the person image IMG from the camera 1. Furthermore, the obtaining unit 211 obtains an infection information relating to the infected person. The authentication unit 212 authenticates the target person included in the person image IMG by using the person image IMG obtained by the information obtaining unit 211. For example, the authentication unit 212 may authenticate the target person that newly enter the stay area PS that corresponds to the camera 1. For example, the authentication unit 212 may authenticate the target person that leaves the stay area PS that corresponds to the camera 1. Furthermore, the authentication unit 212 stores an authentication history information relating to an history of the authentication of the target person in a authentication history DB 221. The contact person identification unit 213 identifies the contact person based on the infection information obtained by the obtaining unit 211 and the authentication history DB 221. The information output unit 214 outputs an information relating to at least one of the infected person and the contact person by using the output apparatus 25.

The storage apparatus 22 is configured to store a desired data. For example, the storage apparatus 22 may temporarily store the computer program that is executed by the arithmetic apparatus 21. The storage apparatus 22 may temporarily store a data that is temporarily used by the arithmetic apparatus 21 when the arithmetic apparatus 21 executes the computer program. The storage apparatus 22 may store a data that is stored for a long term by the information processing apparatus 2. Note that the storage apparatus 22 may include at least one of a RAM (Random Access Memory), a ROM (Read Only Memory), a hard disk apparatus, a magneto-optical disc, a SSD (Solid State Drive) and a disk array apparatus. Namely, the storage apparatus 22 may include a non-transitory recording medium.

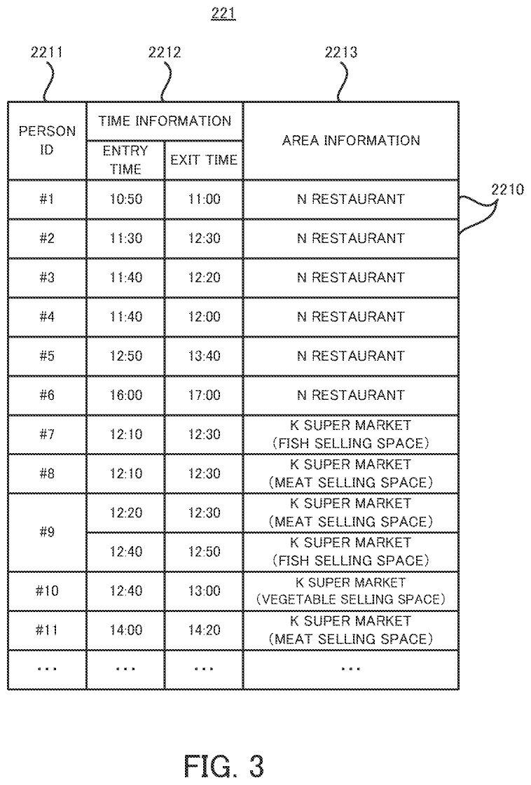

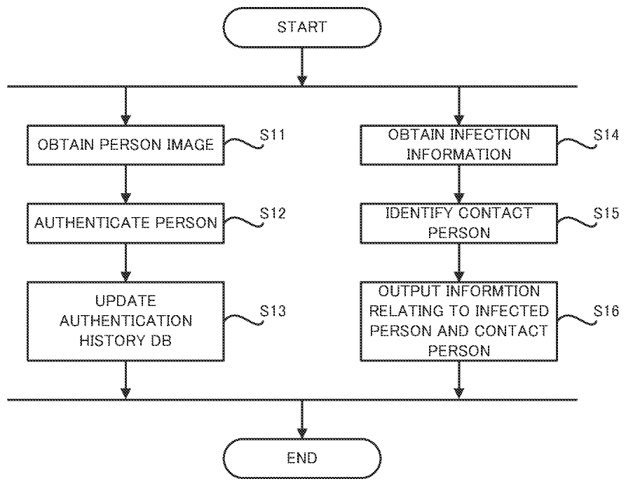

In the first example embodiment, the storage apparatus 22 stores the authentication history DB 221 in which the history information relating to the history of the authentication of the target person by the authentication unit 212 is stored. One example of a data structure of the authentication history DB 221 is illustrated in FIG. 3. As illustrated in FIG. 3, the authentication history DB 221 includes a history record 2210 as the authentication history information relating to the history of the authentication of the target person. The history record 2210 may be generated for each target person authenticated by the authentication unit 212. Thus, the number of the history record 2210 included in the authentication history DB 221 may be equal to the number of the target person authenticated by the authentication unit 212.

The history record 2210 includes a person ID 2211 that is an identifier for identifying the target person, a time information 2212 that indicates a time at which the target person is authenticated and an area information 2213 that indicates the stay area at which the target person is authenticated (namely, the stay area corresponding to the camera 1 that captures the image of the target person). In an example illustrated in FIG. 3, the time information 2212 includes an information that indicates a time at which the target person that enters the stay area is authenticated (namely, a time at which the target person enters the stay area) and an information that indicates a time at which the target person that leaves the stay area is authenticated (namely, a time at which the target person leaves the stay area). In this case, it can be said that the time information 2212 indicates a period during which the target person stays in the stay area.

Note that the authentication history DB 221 may be regarded to indicate a behavior history of the target person. Namely, the authentication history DB 221 may be regarded to store a behavior history information relating to the behavior history of the target person.

Again in FIG. 2, the communication apparatus 23 is configured to communicate with the cameras 1 through the communication network 3. In the first example embodiment, the communication apparatus 23 receives (namely, obtains) the person image IMG from the cameras 2 through the communication network 3.

The input apparatus 24 is an apparatus that receives an input of an information from an outside of the information processing apparatus 2 to the information processing apparatus 2. For example, the input apparatus 24 may include an operational apparatus (for example, at least one of a keyboard, a mouse and a touch panel) that is operable by an operator of the information processing apparatus 2. For example, the input apparatus 24 may include a reading apparatus that is configured to read an information recorded as a data in a recording medium that is attachable to the information processing apparatus 2.

The output apparatus 25 is an apparatus that outputs an information to an outside of the information processing apparatus 2. For example, the output apparatus 25 may output the information as an image. Namely, the output apparatus 25 may include a display apparatus (what we call a display) that is configured to display the image representing the information to be outputted. For example, the output apparatus 25 may output the information as a sound. Namely, the output apparatus 25 may include an audio apparatus (what we call a speaker) that is configured to output the sound. For example, the output apparatus 25 may output the information on a paper. Namely, the output apparatus 25 may include a print apparatus (what we call a printer) that is configured to print a desired information on the paper.

### (1-2) Contact Person Identification Operation Performed by Information Processing Apparatus 2

Next, with reference to FIG. 4, a flow of the contact person identification operation that is performed by the information processing apparatus 2 in the first example embodiment will be described. FIG. 4 is a flowchart that illustrates the flow of the contact person identification operation that is performed by the information processing apparatus 2 in the first example embodiment.

As illustrated in FIG. 4, the information obtaining unit 211 obtains the person image IMG from the camera 1 (a step S11). Then, the authentication unit 212 authenticates the target person that is included in the person image IMG obtained at the step S11 (a step S12). For example, the authentication unit 212 may authenticate the target person by performing a face authentication based on the person image IMG obtained at the step S11. For example, the authentication unit 212 may authenticate the target person by performing an iris authentication based on the person image IMG obtained at the step S11. Then, the authentication unit 212 updates the authentication history DB 221 by using a result of the authentication at the step S12 (a step S13). Namely, authentication unit 212 adds, to the history record 2210 corresponding to the target person authenticated at the step S12, the time information 2212 that indicates the time at which the target person is authenticated at the step S12 and the area information 2213 that indicates the stay area at which the target person is authenticated at the step S12.

In parallel with the processing from the step S11 to the step S13, the contact person identification unit 213 obtains the infection information relating to the infected person (a step S14). For example, the contact person identification unit 213 may obtain the infection information from a server (namely, a server outside the information processing apparatus 2) that manages the infection information relating to the infected person.

The infection information may include an information that is capable of determining the infected person. For example, the infection information may include an information that is capable of determining the infected person by using the person ID 2211 included in the authentication history DB 221. Namely, the infection information may include an information indicating the person ID 2211. For example, the infection information may include an information relating to the stay area in which the infected person stays. For example, the infection information may include an information relating to a time period during which the infected person stays in the stay area.

Then, the contact person identification unit 213 identifies the contact person that possibly contacts the infected person from the plurality of target person authenticated by the authentication unit 212 based on the infection information obtained at the step S14 and the authentication history DB 221 (a step S15). Next, an operation for identifying the contact person based on the infection information indicating that the target person whose person ID 2211 is “#3” and the target person whose person ID 2211 is “#9” are the infected persons and the authentication history DB 221 illustrated in FIG. 3 will be described as one example of the operation for identifying the contact person. Incidentally, in the below described description, the target person whose person ID 2211 is “#K (note that K is an integer that is equal to or larger than 1)” is referred to as the “target person #K”.

The contact person identification unit 213 may identify, as the contact person, at least one target person staying in the stay area in which the infected person stays (hereinafter, this area is referred to as a “closed contact area”) in at least a part of a time period during which the infected person stays in the closed contact area (hereinafter, this time period is referred to as a “closed contact time period”) from the plurality of target persons. Namely, the contact person identification unit 213 may identify, as the contact person, at least one target person staying in the stay area that is same as the closed contact area in a time period that overlaps with the closed contact time period at least partially from the plurality of target persons.

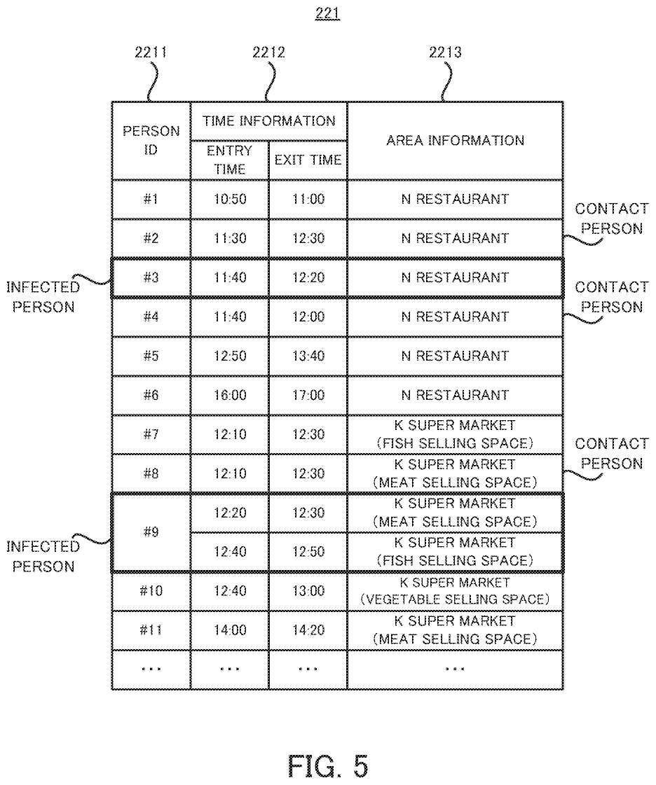

For example, as illustrated in FIG. 5, the target person #3 that is the infected person stays in the stay area that is a N restaurant in a time period from 11:40 to 12:20. In this case, the stay area that is the N restaurant is the closed contact area and the time period from 11:40 to 12:20 is the closed contact time period. As a result, the contact person identification unit 213 may identify, as the contact persons that possibly contact the target person #3 that is the infected person, the target person #2 and the target person #4 staying in the closed contact area that is the N restaurant in at least a part of the closed contact time period from 11:40 to 12:20.

Furthermore, as illustrated in FIG. 5, the target person #9 that is the infected person stays in the stay area that is a meat selling space of a K supermarket in a time period from 12:20 to 12:30. In this case, the stay area that is the meat selling space of the K supermarket is the closed contact area and the time period from 12:20 to 12:30 is the closed contact time period. As a result, the contact person identification unit 213 may identify, as the contact person that possibly contacts the target person #9 that is the infected person, the target person #8 staying in the closed contact area that is the meat selling space of the K supermarket in at least a part of the closed contact time period from 12:20 to 12:30.

Furthermore, as illustrated in FIG. 5, the target person #9 that is the infected person stays in the stay area that is a fish selling space of a K supermarket in a time period from 12:40 to 12:50. In this case, the stay area that is the fish selling space of the K supermarket is the closed contact area and the time period from 12:40 to 12:50 is the closed contact time period. However, as illustrated in FIG. 5, there is no target person staying in the closed contact area that is the fish selling space of the K supermarket in at least a part of the closed contact time period from 12:40 to 12:50. Thus, the contact person identification unit 213 may not identify the target person staying in the closed contact area that is the fish selling space of the K supermarket in at least a part of the closed contact time period from 12:40 to 12:50.

The contact person identification unit 213 may identify, as the contact person, at least one target person staying in the closed contact area in which the infected person stays in at least a part of a time period before a predetermined first time elapses from a time at which the infected person leaves the closed contact area (namely, a time period that is near the closed contact time period, hereinafter, it is referred to as a “near time period”) from the plurality of target persons. Namely, the contact person identification unit 213 may identify, as the contact person, at least one target person staying in the stay area that is same as the closed contact area in a time period that overlaps with the near time period at least partially from the plurality of target persons.

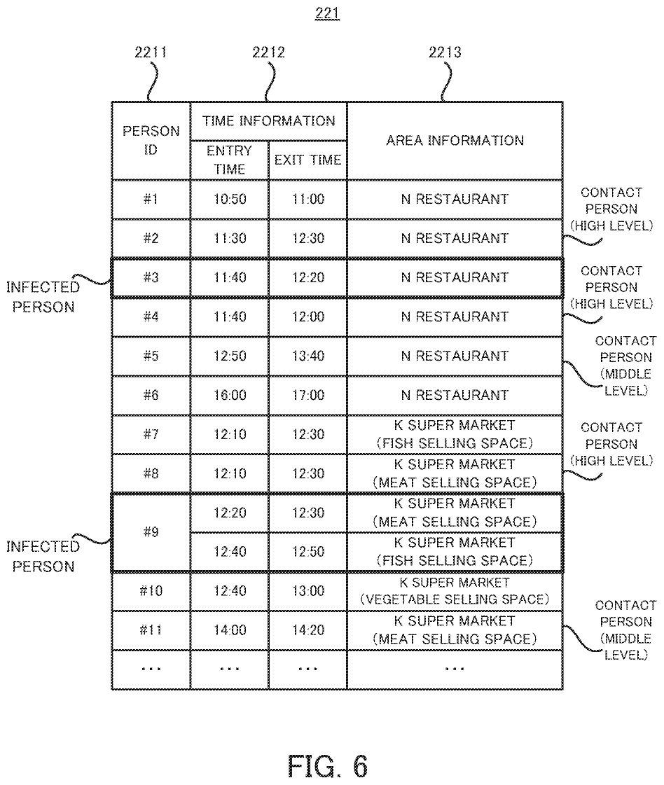

For example, as illustrated in FIG. 6, the target person #3 that is the infected person leaves the closed contact area that is the N restaurant at 12:20. In this case, a time period before the predetermined first time elapses from 12:20 is the near time period. For example, when the first time is set to be a “two hours”, a time period from 12:20 to 14:20 is the near time period. As a result, the contact person identification unit 213 may identify, as the contact person that possibly contacts the target person #3 that is the infected person (specifically, the contact person that is possibly affected by the infection disease which the target person #3 catches), the target person #5 staying in the closed contact area that is the N restaurant in at least a part of the near time period from 12:20 to 14:20.

Furthermore, as illustrated in FIG. 6, the target person #9 that is the infected person leaves the closed contact area that is the meat selling space of the K supermarket at 12:30. In this case, a time period before the predetermined first time elapses from 12:30 is the near time period. For example, when the first time is set to be a “two hours”, a time period from 12:30 to 14:30 is the near time period. As a result, the contact person identification unit 213 may identify, as the contact person that possibly contacts the target person #9 that is the infected person (specifically, the contact person that is possibly affected by the infection disease which the target person #9 catches), the target person #11 staying in the closed contact area that is the meat selling space of the K supermarket in at least a part of the near time period from 12:30 to 14:30.

Furthermore, as illustrated in FIG. 6, the target person #9 that is the infected person leaves the closed contact area that is the fish selling space of the K supermarket at 12:50. In this case, a time period before the predetermined first time elapses from 12:50 is the near time period. For example, when the first time is set to be a “two hours”, a time period from 12:50 to 14:50 is the near time period. However, as illustrated in FIG. 6, there is no target person staying in the closed contact area that is the fish selling space of the K supermarket in at least a part of the near time period from 12:50 to 14:50. Thus, the contact person identification unit 213 may not identify the target person staying in the closed contact area that is the fish selling space of the K supermarket in at least a part of the near time period from 12:50 to 14:50.

When the target person staying in the closed contact area in at least a part of the near time period is identified as the contact person, the contact person identification unit 213 may set a contact level that is an index value for indicating a degree of the contact of the contact person to the infected person. The contact level may be the index value that becomes higher as the degree of the contact of the contact person to the infected person becomes higher.

For example, the contact person identification unit 213 may set the contact level so that the contact level of the target person staying in the closed contact area in at least a part of the closed contact time period is higher than the contact level of the target person staying in the closed contact area in at least a part of the near time period. Namely, the contact person identification unit 213 may set the contact level of the target person staying in the closed contact area in at least a part of the closed contact time period to be a high level and set the contact level of the target person staying in the closed contact area in at least a part of the near time period to be a middle level that indicates that the degree of the contact is lower than that of the high level. Specifically, as illustrated in FIG. 6, the contact person identification unit 213 may set the contact level of each of the target persons #2 and #4 staying in the closed contact area that is the N restaurant in at least a part of the closed contact time period from 11:40 to 12:20 to be the high level and set the contact level of the target person #5 staying in the closed contact area that is the N restaurant in at least a part of the near time period from 12:20 to 14:20 to be the middle level. As illustrated in FIG. 6, the contact person identification unit 213 may set the contact level of the target person #8 staying in the closed contact area that is the meat selling space of the K supermarket in at least a part of the closed contact time period from 12:20 to 12:30 to be the high level and set contact level of the target person #11 staying in the closed contact area that is the meat selling space of the K supermarket in at least a part of the near time period from 12:50 to 14:50 to be the middle level.

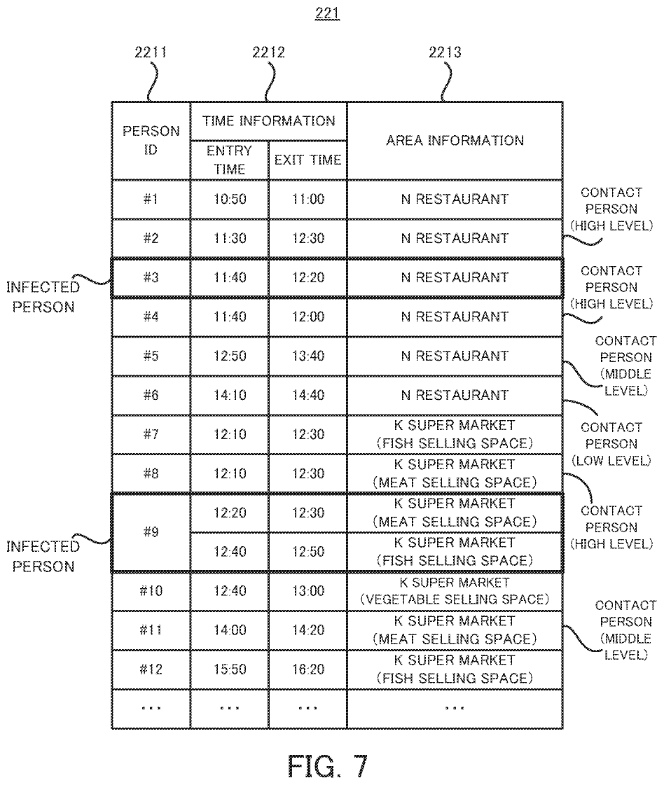

For example, the contact person identification unit 213 may set the contact level so that the contact level of the target person staying in the closed contact area before a predetermined second time elapses from the time at which the infected person leaves the closed contact area is higher than the contact level of the target person staying in the closed contact area after the predetermined second time elapses from the time at which the infected person leaves the closed contact area. Namely, the contact person identification unit 213 may set the contact level of the target person staying in the closed contact area before the predetermined second time elapses from the time at which the infected person leaves the closed contact area to be the middle level and set the contact level of the target person staying in the closed contact area after the predetermined second time elapses from the time at which the infected person leaves the closed contact area to be a low level that indicates that the degree of the contact is lower than that of the middle level. In other words, the contact person identification unit 213 may set the contact level so that the contact level becomes higher as an elapsed time from the time at which the infected person leaves the closed contact area to a time at which the contact person starts to stay in the closed contact area becomes shorter. Specifically, as illustrated in FIG. 7, the contact person identification unit 213 may set the contact level of the target person #5 staying in the closed contact area that is the N restaurant before one hour elapses from 12:20 that is the time at which the target person #3 that is the infected person leaves the closed contact area to be the middle level and set the contact level of the target person #6 staying in the closed contact area that is the N restaurant after one hour elapses from 12:20 that is the time at which the target person #3 that is the infected person leaves the closed contact area to be the low level.

Note that the contact level is not limited to three levels including the high level, the middle level and the low level. The contact person identification unit 213 may set the contact level to be at least one of any number of levels.

Moreover, the contact person identification unit 213 may calculate a contact score that is an index value for quantitatively indicating the degree of the contact of the contact person to the infected person, in addition to or instead of setting the contact level of the contact person. The contact score may be the index value that becomes larger as the degree of the contact of the contact person to the infected person becomes higher. Moreover, when the contact score is calculated, the contact person identification unit 213 may rank the plurality of contact persons based on the contact score. For example, the contact person identification unit 213 may generate a list in which the plurality of contact persons are listed in a descending order of the degree of the contact to the infected person (for example, in a descending order of the contact score).

The contact person identification unit 213 may identify, as the contact person, at least one target person staying in an area that is near the closed contact area in which the infected person stays (hereinafter, this area is referred to as a “near area”) in at least a part of the closed contact time period from the plurality of target persons. Namely, the contact person identification unit 213 may identify, as the contact person, at least one target person staying in the near area that is different from the closed contact area but is near the closed contact area in a time period that overlaps with the closed contact time period at least partially from the plurality of target persons.

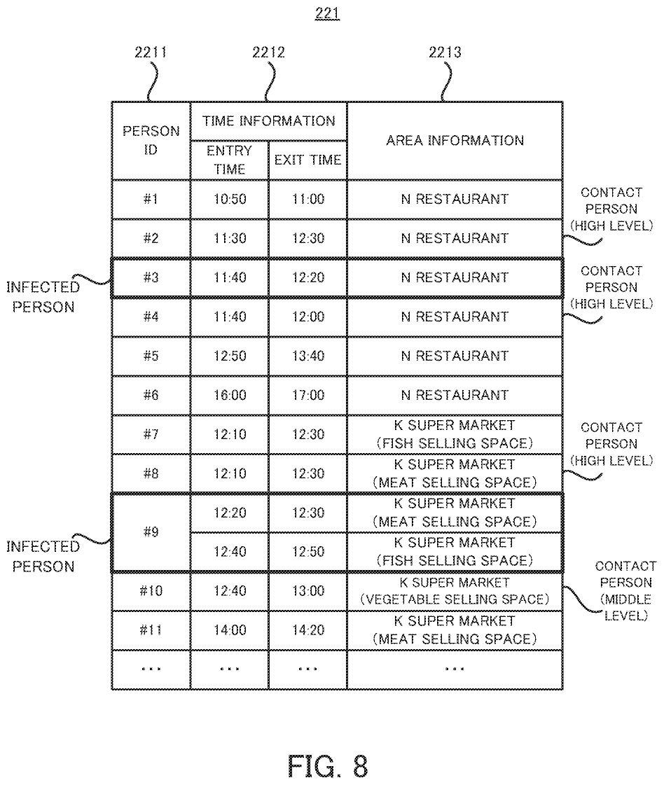

The near area that is near a certain stay area may be set in advance. As one example, an operation for identifying the contact person when the stay area that is a vegetable selling space of the K supermarket is set as the near area that is near the stay area that is the fish selling space of the K supermarket will be described with reference to FIG. 8. In this case, as illustrated in FIG. 8, the target person #9 that is the infected person stays in the closed contact area that is the fish selling space of the K supermarket in the closed contact time period from 12:40 to 12:50. In this case, the contact person identification unit 213 may identify, as the contact person that possibly contacts the target person #9 that is the infected person (specifically, the contact person that is possibly affected by the infection disease which the target person #9 catches), the target person #10 staying in the near area that is the vegetable selling space of the K supermarket in at least a part of the closed contact time period from 12:40 to 12:50.

When the target person staying in the near area is identified as the contact person, the contact person identification unit 213 may set the contact level so that the contact level of the target person staying in the closed contact area in at least a part of the closed contact time period is higher than the contact level of the target person staying in the near area in at least a part of the closed contact time period. Namely, the contact person identification unit 213 may set the contact level of the target person staying in the closed contact area in at least a part of the closed contact time period to be the high level and set the contact level of the target person staying in the near area in at least a part of the closed contact time period to be the middle level or the low level that indicates that the degree of the contact is lower than that of the high level. Specifically, as illustrated in FIG. 8, the contact person identification unit 213 may set the contact level of the target person #8 staying in the closed contact area that is the fish selling space of the K supermarket in at least a part of the closed contact time period from 12:40 to 12:50 to be the high level and set the contact level of the target person #10 staying in the near area that is the vegetable selling space of the K supermarket in at least a part of the closed contact time period from 12:40 to 12:50 to be the middle level or the low level.

Again in FIG. 4, then, the information output unit 214 outputs the information relating to at least one of the infected person and contact person by using the output apparatus 25 (a step S16). For example, the information output unit 214 may output, to the contact person identified at the step S15, an information for notifying that he possibly contacts the infected person. For example, the information output unit 214 may output, to the contact person identified at the step S15, an information for encouraging him to go to a medical institution for an inspection. For example, the information output unit 214 may output, to the contact person identified at the step S15, an information for encouraging him to self-isolate to prevent an infection to a person around the contact person. For example, the information output unit 214 may output, to a concerned person of the stay area (for example, a worker and the like of the store) in which at least one of the infected person and the contact person stays, an information relating to the stay area in which at least one of the infected person and the contact person stays. For example, the information output unit 214 may output, to the concerned person of the stay area (for example, the worker and the like of the store) in which at least one of the infected person and the contact person stays, an information for encouraging him to clean the stay area in which at least one of the infected person and the contact person stays. For example, the information output unit 214 may output, to the concerned person of the stay area (for example, the worker and the like of the store) in which at least one of the infected person and the contact person stays, an information relating to the concerned person that possibly contacts at least one of the infected person and the contact person (for example, the concerned person that deals with at least one of the infected person and the contact person (for example, used a cash register)).

The information output unit 214 may output the information relating to at least one of the infected person and contact person by using the communication apparatus 23 in addition to or instead of the output apparatus 25. For example, the information output unit 214 may transmit the information relating to at least one of the infected person and contact person to a mobile terminal of a person that is a target for outputting the information by using the communication apparatus 23. As one example, the information output unit 214 may transmit a text message for notifying the information relating to at least one of the infected person and contact person to the mobile terminal of the person that is the target for outputting the information by using the communication apparatus 23. As one example, the information output unit 214 may transmit an audio message for notifying the information relating to at least one of the infected person and contact person to the mobile terminal of the person that is the target for outputting the information by using the communication apparatus 23.

### (1-3) Technical Effect of Information Processing System SYSa

As described above, in the first example embodiment, the information processing apparatus 2 is capable of determining the contact person based on the authentication history DB 221 corresponding to the behavior history information that is generated based on the person image IMG generated by means of the camera 1 capturing the image of the target person. Thus, the information processing apparatus 2 is capable of identifying, as the contact person, the target person that is not so close to the infected person that the information can be receivable from the mobile terminal of the infected person and/or the information can be transmittable to the mobile terminal of the infected person but is possibly affected by the infection disease (for example, that possibly contacts the object to which the airborne droplet of the infected person is adhered). Namely, the information processing apparatus 2 is capable of solving the technical problem of the behavior management system disclosed in the above described Patent Literature 1. Thus, in the information processing apparatus 2, there is a lower possibility that a situation in which the target person that should be identified as the contact person is not identified as the contact person occurs.

Furthermore, the information processing apparatus 2 is capable of identifying, as the contact person, the target person staying in the closed contact area in at least a part of not only the closed contact time period but also the near time period. Thus, the information processing apparatus 2 is capable of properly identifying, as the contact person, the target person that does not directly contact the infected person but is possibly affected by the infection disease.

Furthermore, the information processing apparatus 2 is capable of setting the contact level so that the contact level of the target person staying in the closed contact area in at least a part of the closed contact time period is higher than the contact level of the target person staying in the closed contact area in at least a part of the near time period. Thus, the information processing apparatus 2 is capable of indirectly encouraging an operator that executes a countermeasure for the infection disease to execute the counter measure for the infection disease based on the contact level.

Furthermore, the information processing apparatus 2 is capable of setting the contact level so that the contact level becomes higher as the elapsed time from the time at which the infected person leaves the closed contact area to the time at which the contact person starts to stay in the closed contact area becomes shorter. Thus, the information processing apparatus 2 is capable of indirectly encouraging the operator that executes the countermeasure for the infection disease to execute the counter measure for the infection disease based on the contact level.

Furthermore, the information processing apparatus 2 is capable of identifying, as the contact person, the target person staying in not only the closed contact area but also the near area. Thus, the information processing apparatus 2 is capable of properly identifying, as the contact person, the target person that does not directly contact the infected person but is possibly affected by the infection disease.

Furthermore, the information processing apparatus 2 is capable of setting the contact level so that the contact level of the target person staying in the closed contact area is higher than the contact level of the target person staying in the near area. Thus, the information processing apparatus 2 is capable of indirectly encouraging the operator that executes the countermeasure for the infection disease to execute the counter measure for the infection disease based on the contact level.

### (1-4) Modified Example of Information Processing System SYSa

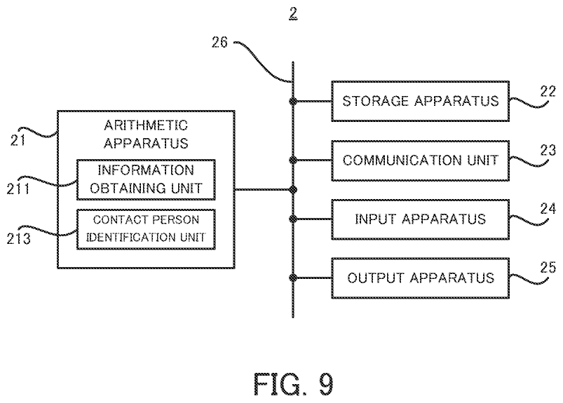

As illustrated in FIG. 9, the information processing apparatus 2 may not include the authentication unit 212. In this case, the information processing apparatus 2 may generate a behavior history DB relating to the behavior history of the target person included in the person image IMG, instead of the authentication history DB 221. The behavior history DB may include the history record 2210 as with the authentication history DB 221. However, when the behavior history DB is generated, the person ID 2211 may be an identifier for identifying the person included in the person image IMG, the time information 2212 may indicate a time at which the person is captured in the person image IMG, and the area information 2213 may indicate the stay area in which the person included in the person image IMG stays (namely, the stay area corresponding to the camera 1 that captures the image of the person). As a result, even when the information processing apparatus 2 does not include the authentication unit 212, the information processing apparatus 2 is capable of identifying the contact person.

Alternatively, when the information processing apparatus 2 does not include the authentication unit 212, the information processing apparatus 2 (especially, the storage apparatus 22) may not store the authentication history DB221 and the behavior history DB. In this case, the information processing apparatus 2 may identify the contact person based on at least one of the authentication history DB221 and the behavior history DB stored in an server outside the information processing apparatus 2.

As illustrated in FIG. 9, the information processing apparatus 2 may not include the information output unit 214. Namely, the information processing apparatus 2 may not output the information relating to at least one of the infected person and the contact person.

### (2) Information Processing System SYS in Second Example Embodiment

Next, the information processing system SYS in a second example embodiment will be described. Incidentally, in the below described description, the information processing system SYS in the second example embodiment is referred to as the “information processing system SYSb”.

### (2-1) Configuration of Information Processing System SYSb

(2-1-1) Entire Configuration of Information Processing System SYSb

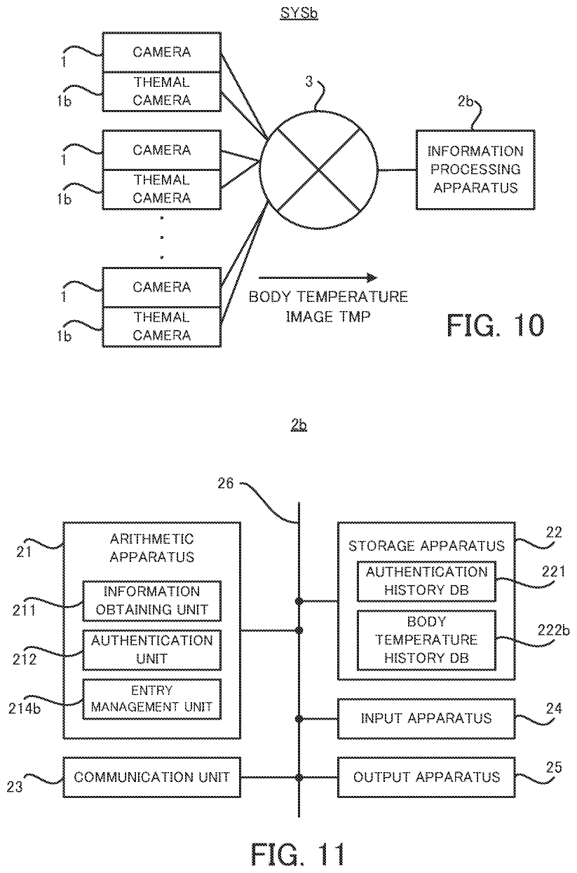

Firstly, with reference to FIG. 10, an entire configuration of the information processing system SYSb in the second example embodiment will be described. FIG. 10 is a block diagram that illustrates the entire configuration of the information processing system SYSb in the second example embodiment. Note that a detailed description of the component that is already described is omitted by assigning the same reference number thereto.

As illustrated in FIG. 10, the information processing system SYSb in the second example embodiment is different from the above described information processing system SYSa in the first example embodiment in that it further includes thermal cameras 1b. The information processing system SYSb is further different from the information processing system SYSa that it includes an information processing apparatus 2b instead of the information processing apparatus 2. Another feature of the information processing system SYSb may be same as another feature of the information processing system SYSa.

The information processing system SYSb includes the plurality of thermal cameras 1b, however, may include a single thermal camera 1b. The thermal cameras 1b and the information processing apparatus 2 may be configured to communicate with each other through the communication network 3.

The thermal camera 1b is an imaging apparatus that is configured to capture an image of the target person (namely, the person) that is located in an imaging target range of the thermal camera 1b. Incidentally, in the below described description, the target person the image of which is captured by the thermal camera 1b may be referred to as a captured person. The thermal camera 1b generates a thermal image TMP that indicates a body temperature of the target person the image of which is captured by the thermal camera 1b by capturing the image of the target person. The thermal image TMP that indicates the body temperature of the target person may be typically an image in which the target person is substantially included by a distribution of the body temperature of the target person. Note that the “thermal image TMP in which the target person is included” may include an image that is generated by means of the thermal camera 1b capturing the image of the target person that does not have an intention of wanting the thermal camera 1b to capture the image of the target person. The “thermal image TMP in which the target person is included” may include an image that is generated by means of the thermal camera 1b capturing the image of the target person that has the intention of wanting the thermal camera 1b to capture the image of the target person. The thermal camera 1b outputs the generated thermal image TMP to the information processing apparatus 2b. Specifically, the thermal camera 1b transmits the generated thermal image TMP to the information processing apparatus 2b through the communication network 3.

In the first example embodiment, the thermal camera 1b is placed at at least one of the plurality of stay areas in which the target person can stay. Typically, the plurality of thermal cameras 1b may be placed at the plurality of different stay areas, respectively. Namely, each thermal camera 1b may be placed at one stay area that corresponds to each thermal camera 1b. In this case, the thermal camera 1b may capture the image of the target person that newly enters the stay area from an area that is different from the stay area. The thermal camera 1b may capture the image of the target person that leaves the stay area to the area that is different from the stay area. The thermal camera 1b may capture the image of the target person that is staying in the stay area.

Note that the plurality of thermal cameras 1b may be respectively placed at all of the plurality of stay areas in which the plurality of cameras 1 are respectively placed. Alternatively, the thermal camera 1b may be placed at at least one of the plurality of stay areas in which the plurality of cameras 1 are respectively placed and the thermal camera 1b may not be placed at at least another one of the plurality of stay areas in which the plurality of cameras 1 are respectively placed. Alternatively, the thermal camera 1b may be placed at the stay area in which the camera 1 is not placed.

When the camera 1 and the thermal camera 1b are placed at a certain stay area, the imaging target range of the camera 1 may overlap with the imaging target range of the thermal camera 1b at least partially. In this case, the thermal camera 1b may capture the image of the target person the image of which is captured by the camera 1. The camera 1 may capture the image of the target person the image of which is captured by the thermal camera 1b. However, the imaging target range of the camera 1 may not overlap with the imaging target range of the thermal camera 1b.

The information processing apparatus 2b receives the thermal image TMP that is transmitted from the thermal camera 1b through the communication network 3. The information processing apparatus 2b performs an entry management operation for managing an entry of the target person to the stay area by using the received thermal image TMP. Specifically, in the second example embodiment, the stay area is set to be a restriction area which the target person is not permitted to enter unless the body temperature of the target person is measured at a timing at which the target person wants to enter the stay area. Furthermore, the stay area is set to be the restriction area which the target person is not permitted to enter unless the measured body temperature is lower than an allowable threshold value even when the body temperature of the target person is measured at the timing at which the target person wants to enter the stay area. Thus, the information processing apparatus 2b obtains the thermal image TMP from the thermal camera 1b that is placed at the stay area at the timing at which the target person wants to enter the stay area. Furthermore, the information processing apparatus 2b calculates the body temperature of the target person based on the thermal image TMP and permits the target person to enter the stay area when the calculated body temperature is lower than the allowable threshold value. For example, when a gate apparatus through which the target person should pass to enter the stay area is placed, the information processing apparatus 2b may permit the target person to enter the stay area by setting a state of the gate apparatus to be an open state in which the target person can pass through the gate apparatus. On the other hand, when the calculated body temperature is higher than the allowable threshold value, the target person is not permitted to enter the stay area. For example, when the gate apparatus through which the target person should pass to enter the stay area is placed, the information processing apparatus 2b may not permit the target person to enter the stay area by setting the state of the gate apparatus to be the close state in which the target person cannot pass through the gate apparatus.

(2-1-2) Configuration of Information Processing Apparatus 2b

Next, with reference to FIG. 11, a configuration of the information processing apparatus 2b in the second example embodiment will be described. FIG. 11 is a block diagram that illustrates the configuration of the information processing apparatus 2b in the second example embodiment.

As illustrated in FIG. 11, the information processing apparatus 2b in the second example embodiment is different from the above described information processing apparatus 2 in the first example embodiment in that it may not include the contact person identification unit 213 and the information output unit 214. However, the information processing apparatus 2b may include at least one of the contact person identification unit 213 and the information output unit 214. Furthermore, the information processing apparatus 2b is different from the information processing apparatus 2 in that it includes an entry management unit 214b that is one specific example of a “permitting unit” as the logical function block implemented in the arithmetic apparatus 21. Furthermore, the information processing apparatus 2b is different from the information processing apparatus 2 in that the storage apparatus 22 stores a body temperature history DB 222b. Another feature of the information processing apparatus 2b may be same as another feature of the information processing apparatus 2.

The entry management unit 214b performs the entry management operation for managing the entry of the target person to the stay area by using the received thermal image TMP. Furthermore, the entry management unit 214b calculates the body temperature of the target person based on the thermal image TMP and updates the body temperature DB 222b that indicates a history of the calculated body temperature.

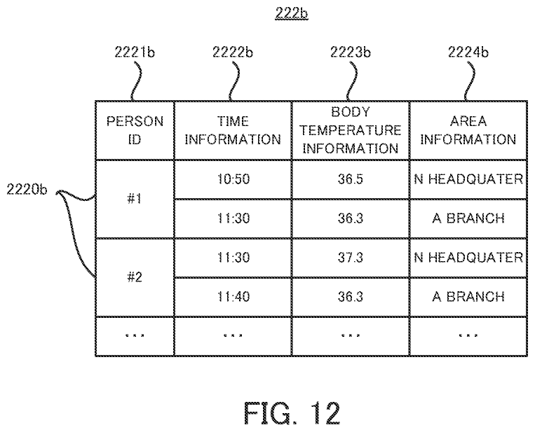

One example of a data structure of the body temperature history DB 222b is illustrated in FIG. 12. As illustrated in FIG. 12, the body temperature history DB 222b includes a history record 2220b as a body temperature history information relating to the history of the body temperature of the target person. The history record 2220b may be generated for each target person the body temperature of which is measured. Thus, the number of the history record 2220b included in the body temperature history DB 222b may be equal to the number of the target person the body temperature of which is measured.

The history record 2220b includes a person ID 2221b that is an identifier for identifying the target person, a time information 2222b that indicates a time at which the body temperature of the target person is measured, a body temperature information 2223b that indicates the body temperature of the target person and an area information 2224b that indicates the stay area at which the body temperature of the target person is measured.

(2-2) Entry Management Operation performed by Information Processing Apparatus 2b

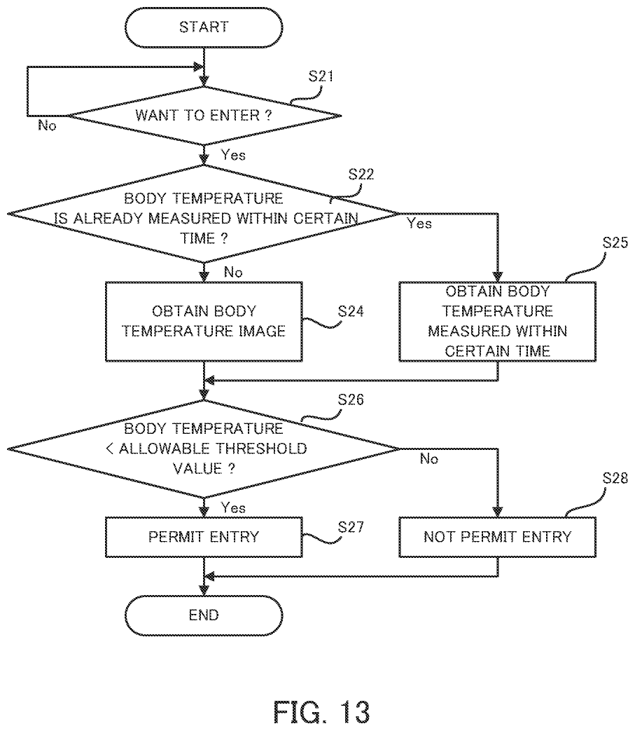

Next, with reference to FIG. 13, a flow of the entry management operation that is performed by the information processing apparatus 2b in the second example embodiment will be described. FIG. 13 is a flowchart that illustrates the flow of the entry management operation that is performed by the information processing apparatus 2b in the second example embodiment.

As illustrated in FIG. 13, the entry management unit 214b determines whether or not the target person wants to enter the stay area (a step S21). For example, as described in the first example embodiment, the authentication unit 212 authenticates the target person based on the person image IMG when the target person enters the stay area. Thus, the entry management unit 214b may determine that the target person wants to enter the stay area when the information obtaining unit 211 obtains the person image IMG that is used to authenticate the target person. Alternatively, the entry management unit 214b may determine that the target person wants to enter the stay area when an information for notifying that the target person wants to enter the stay area is obtained by using the communication apparatus 23 or the input apparatus 24.

As a result of the determination at the step S21, when it is determined that the target person wants to enter the stay area (the step S21: Yes), the entry management unit 214b determines by using the body temperature history DB 222b whether or not the body temperature of the target person that wants to enter the stay area (hereinafter, it is referred to as an “entry requesting person”) is already measured in the past (a step S22). Specifically, the entry management unit 214b determines the history record 2220b that corresponds to the entry requesting person and determines based on the history record 2220b whether or not the body temperature of the entry requesting person is already measured. Especially, the entry management unit 214b determines whether or not the body temperature of the entry requesting person is already measured in a past period within a given time (for example, one hour) from a time at which it is determined that the target person wants to enter the stay area.

As a result of the determination at the step S21, when it is determined that the body temperature of the entry requesting person is not already measured (the step S22: No), it is estimated that the given time already elapses after the body temperature of the entry requesting person is measured although the body temperature of the entry requesting person is already measured in the past. In this case, there is a relatively low possibility that the current body temperature of the entry requesting person is about same as the body temperature that is already measured in the past. Alternatively, when it is determined that the body temperature of the entry requesting person is not already measured, there is a possibility that the body temperature of the entry requesting person is not measured at all in the past. Thus, in this case, the information obtaining unit 211 newly obtains the thermal image TMP from the thermal camera 1b that is placed at the stay area which the entry requesting person wants to enter (a step S24).

Note that the thermal cameras 1b are not always placed at all of the stay areas as described above. Thus, when the thermal camera 1b is not placed at the stay area which the entry requesting person wants to enter, the entry management unit 214b may present, to the entry requesting person, a message that encourages to measure the body temperature at the stay area at which the thermal camera 1b is placed. For example, the entry management unit 214b may present the message that encourages to measure the body temperature together with an information that indicates a route from a current position of the entry requesting person (namely, the stay area which the entry requesting person wants to enter) to another stay area at which the thermal camera 1b is placed.

Then, the entry management unit 214b calculates the body temperature of the entry requesting person based on the thermal image TMP and determines whether or not the calculated body temperature is lower than the allowable threshold value (a step S26). Note that the entry management unit 214b may update the body temperature history DB 222b by using an information relating to the body temperature calculated from the thermal image TMP when the thermal image TMP is obtained. As a result of the determination at the step S26, when it is determined that the calculated body temperature is lower than the allowable threshold value (the step S26: Yes), the entry management unit 214b permits the entry requesting person to enter the stay area (a step S27). On the other hand, as a result of the determination at the step S26, when it is determined that the calculated body temperature is higher than the allowable threshold value (the step S26: No), the entry management unit 214b does not permit the entry requesting person to enter the stay area (a step S28).

On the other hand, as a result of the determination at the step S21, when it is determined that the body temperature of the entry requesting person is already measured (the step S22: Yes), it is estimated that the given time does not elapse yet after the body temperature of the entry requesting person is measured. Namely, it is estimated that much time does not elapse yet after the body temperature of the entry requesting person is measured. In this case, there is a relatively high possibility that the current body temperature of the entry requesting person is about same as the body temperature that is already measured in the past. Conversely, a threshold value that is the “given time” used at the step S22 may be set to be a proper value that is allowed to distinguish a state in which there is a relatively high possibility that the current body temperature of the entry requesting person is about same as the body temperature that is already measured in the past from a state in which there is a relatively low possibility that the current body temperature of the entry requesting person is about same as the body temperature that is already measured in the past by an elapsed time after the body temperature of the entry requesting person is measured. In this case, the entry management unit 214b assumes that the current body temperature of the entry requesting person is same as the body temperature that is already measured in the past. Thus, the information obtaining unit 211 may not newly obtain the thermal image TMP. The entry management unit 214b obtains, from the body temperature history DB 222b, the temperature information 2223b indicating the body temperature of the entry requesting person that is already measured in the past (specifically, the body temperature that is already measured within the given time from the time at which it is determined that the target person wants to enter the stay area).

Then, the entry management unit 214b determines whether or not the body temperature indicated by the temperature information 2223b is lower than the allowable threshold value (the step S26). Incidentally, when the body temperature of the entry requesting person is measured a plurality of times (namely, the temperature information 2223b indicates a plurality of body temperatures that are measured at a plurality of different times, respectively), the entry management unit 214b may determine whether or not at least one of the plurality of body temperatures is lower than the allowable threshold value. For example, the entry management unit 214b may determine whether or not the latest body temperature of the plurality of body temperatures is lower than the allowable threshold value. Alternatively, the entry management unit 214b may determine whether or not an average value of the plurality of body temperatures is lower than the allowable threshold value. As a result of the determination at the step S26, when it is determined that the body temperature indicated by the temperature information 2223b is lower than the allowable threshold value (the step S26: Yes), the entry management unit 214b permits the entry requesting person to enter the stay area (the step S27). On the other hand, as a result of the determination at the step S26, when it is determined that the body temperature indicated by the temperature information 2223b is higher than the allowable threshold value (the step S26: No), the entry management unit 214b does not permit the entry requesting person to enter the stay area (the step S28).

### (2-3) Technical Effect of Information Processing System SYSb

As described above, in the second example embodiment, the information processing apparatus 2b may not newly obtain the thermal image TMP when the body temperature of the entry requesting person is already measured within the given time (for example, one hour) from the time at which it is determined that the target person wants to enter the stay area. In this case, the information processing apparatus 2b permits the entry requesting person to enter the stay area when the body temperature that is already measured in the past is lower than the allowable threshold value. This is because there is a relatively low possibility that the body temperature of the entry requesting person changes rapidly in a short time and thus a problem rarely occurs even when the body temperature that is already measured in the past is used as the current body temperature of the entry requesting person. Thus, even when the entry requesting person wants to enter the stay area at which the thermal camera 1b is not placed, the information processing apparatus 2b is capable of restricting the entry of the entry requesting person that possibly catches some disease because the body temperature is higher than the allowable threshold value.

Furthermore, the thermal cameras 1b may not be necessarily placed at all of the stay areas, and thus, an installation cost of the information processing system SYSb is reducible.

### (2-4) Modified Example of Information Processing System SYSb

In the above described description, the information processing system SYSb includes the thermal camera 1b. However, the information processing system SYSb may include a body temperature detection apparatus that is configured to detect the body temperature of the target person in addition to or instead of the thermal cameras 1b.

In the above described description, the information processing system SYSb includes the cameras 1. However, the information processing system SYSb may not include the cameras 1. In this case the information processing system SYSb may not authenticate the target person by using the person image IMG. Moreover, in this case, the information processing system SYSb may not include the authentication unit 212 as described in the first example embodiment.

### (3) Information Processing System SYS in Third Example Embodiment

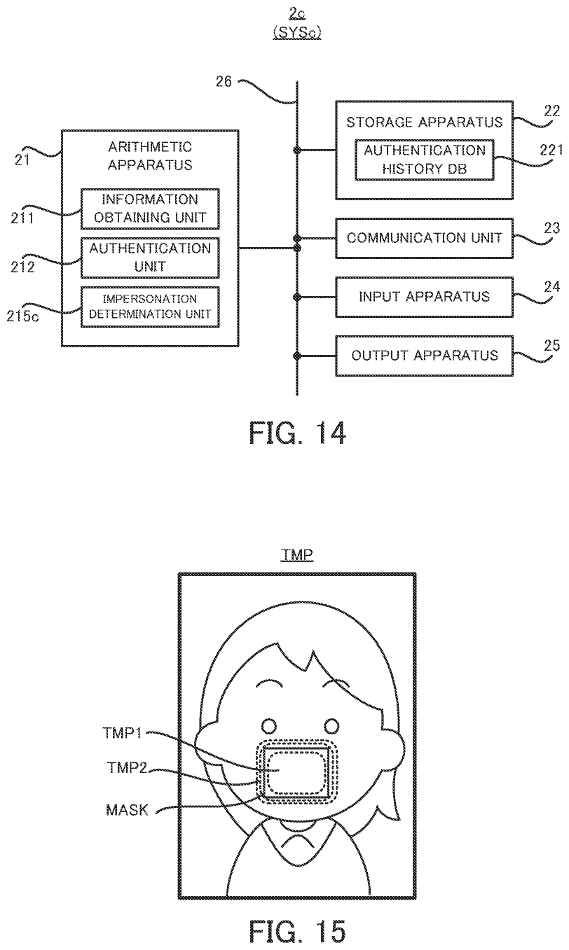

Next, the information processing system SYS in a third example embodiment will be described. Incidentally, in the below described description, the information processing system SYS in the third example embodiment is referred to as the “information processing system SYSc”. The information processing system SYSc in the third example embodiment is different from the above described information processing system SYSb in the second example embodiment in that it includes an information processing apparatus 2c instead of the information processing apparatus 2b. Another feature of the information processing system SYSc may be same as another feature of the information processing system SYSb. Thus, next, with reference to FIG. 14, a configuration of the information processing apparatus 2c in the third example embodiment will be described. FIG. 14 is a block diagram that illustrates the configuration of the information processing apparatus 2c in the third example embodiment.

As illustrated in FIG. 14, the information processing apparatus 2c in the third example embodiment is different from the above described information processing apparatus 2b in the second example embodiment in that it may not include the entry management unit 214b. Namely, the information processing apparatus 2c is different from the information processing apparatus 2b in that it may not perform the entry management operation using the thermal image TMP. Thus, the information processing system SYSc may not store the body temperature history DB 222b. Furthermore, the information processing apparatus 2c is different from the information processing apparatus 2b in that it includes an impersonation determination unit 215c that is one specific example of a “determination unit”. Another feature of the information processing apparatus 2c may be same as another feature of the information processing apparatus 2b.

The impersonation determination unit 215 determines based on the thermal image TMP whether or not the target person that is located in front of the camera 1 impersonates (pretends to be) another person that is different from the target person. Especially, the impersonation determination unit 215 determines based on the thermal image TMP whether or not the target person that is located in front of the camera 1 impersonates another person that is different from the target person, when the target person that is included in the person image IMG generated by the camera 1 wears a mask.

Specifically, the camera 1 usually captures the image of the target person that is located in front of the camera 1. In this case, the thermal camera 1b also captures the image of the target person that is located in front of the camera 1 (namely, in front of the thermal camera 1b). Here, when the target person wears the mask, a temperature of at least a part of a mask part TMP1, which is covered by the mask, of the face of the target person included in the thermal image TMP fluctuates periodically in synchronization with a breathing of the target person, as illustrated in FIG. 15 that illustrates the thermal image TMP. This is because a temperature of an exhaled breath of the target person is relatively high. Moreover, when the target person wears the mask, a temperature of at least a part of an adjacent part TMP2, which is adjacent to an outer edge of the mask (namely, the adjacent part TMP2 that surrounds the mask part TMP1), of the face of the target person included in the thermal image TMP fluctuates periodically in synchronization with a breathing of the target person that is leaked from the mask, as illustrated in FIG. 15.

Note that FIG. 15 illustrates that the face of the target person is clearly included in the thermal image TMP for a visibility of drawing, however, the thermal image TMP is actually an image that has a contrast and/or a color based on the body temperature of the target person.

On the other hand, there is a possibility that the target person that has a malicious intention of impersonating another person makes the camera 1 capture an image of a portable display (for example, a display of a smartphone or a tablet terminal) or a picture in which another person is included. In this case, the thermal camera 1b also capture an image of the display or the picture that is located in front of the camera 1 (namely, in front of the thermal camera 1b). Here, even when another person included in the display or the picture wears the mask, the temperature of at least a part of the mask part TMP1 and the adjacent part TMP2 of the thermal image TMP does not fluctuate periodically in synchronization with a breathing of another person. This is because the thermal image TMP merely indicates not the body temperature of another person included in the display or the picture but a temperature of the display or the picture itself.

Thus, the impersonation determination unit 215c determines whether or not the temperature of at least a part of the mask part TMP1 and the adjacent part TMP2 of the thermal image TMP fluctuates periodically, in order to determine whether or not the target person that is in front of the camera 1 impersonates another person. When the temperature of at least a part of the mask part TMP1 and the adjacent part TMP2 of the thermal image TMP fluctuates periodically, it is estimated that the target person that is in front of the camera 1 is actually included in the person image IMG and the thermal image TMP. Thus, in this case, the impersonation determination unit 215c may determine that the target person that is in front of the camera 1 does not impersonate another person. On the other hand, when the temperature of at least a part of the mask part TMP1 and the adjacent part TMP2 of the thermal image TMP does not fluctuate periodically, it is estimated that another person that is different from the target person in front of the camera 1 (especially, another person included in the display or the picture) is included in the person image IMG and the thermal image TMP. Thus, in this case, the impersonation determination unit 215c may determine that the target person that is in front of the camera 1 impersonates another person.

As described above, in the third example embodiment, the information processing system SYSc is capable of properly determining whether or not the target person that is in front of the camera 1 impersonates another person.

Note that the impersonation determination unit 215c may present, to the target person, a message that encourages to breathe (especially, to blow out a breath). In this case, the impersonation determination unit 215c may determine whether or not the temperature of at least a part of the mask part TMP1 and the adjacent part TMP2 fluctuates periodically in a period during which the message is presented. As a result, the information processing system SYSc is capable of determining whether or not the target person that is in front of the camera 1 impersonates another person more properly.

### (4) Information Processing System SYS in Fourth Example Embodiment

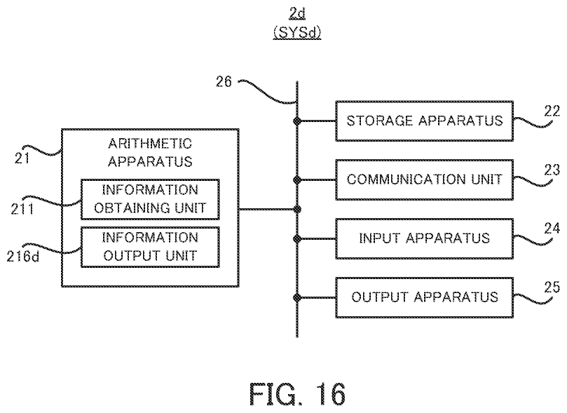

Next, the information processing system SYS in a fourth example embodiment will be described. Incidentally, in the below described description, the information processing system SYS in the fourth example embodiment is referred to as the “information processing system SYSd”. The information processing system SYSd in the fourth example embodiment is different from the above described information processing system SYSb in the second example embodiment in that it includes an information processing apparatus 2d instead of the information processing apparatus 2b. Another feature of the information processing system SYSd may be same as another feature of the information processing system SYSb. Thus, next, with reference to FIG. 16, a configuration of the information processing apparatus 2d in the fourth example embodiment will be described. FIG. 16 is a block diagram that illustrates the configuration of the information processing apparatus 2d in the third example embodiment.

As illustrated in FIG. 14, the information processing apparatus 2d in the fourth example embodiment is different from the above described information processing apparatus 2b in the second example embodiment in that it may not include the entry management unit 214b. Namely, the information processing apparatus 2d is different from the information processing apparatus 2b in that it may not perform the entry management operation using the thermal image TMP. Thus, the information processing system SYSd may not store the body temperature history DB 222b. Furthermore, the information processing apparatus 2d is different from the information processing apparatus 2b in that it may not include the authentication unit 212. Namely, the information processing apparatus 2d is different from the information processing apparatus 2b in that it may not authenticate the target person by using the person image IMG. Thus, the information processing system SYSd may not include the cameras 1 and may not store the body temperature history DB 222b. Furthermore, the information processing apparatus 2d is different from the information processing apparatus 2b in that it includes an information output unit 216d that is one specific example of an “outputting unit”. Another feature of the information processing apparatus 2d may be same as another feature of the information processing apparatus 2b.

In the fourth example embodiment, at least one thermal camera 1b is placed in a restaurant that is one example of the stay area. In this case, the thermal camera 1b captures the image of the target person staying in the restaurant (namely, a customer of the restaurant).

The information output unit 216d determines whether or not the target person included in the thermal image TMP wears the mask based on the thermal image TMP. For example, the information output unit 216d may determine whether or not the target person included in the thermal image TMP wears the mask based on a difference between the body temperature of the face included in the thermal image TMP and a temperature of the mask included in the thermal image TMP.

Furthermore, when it is determines that the target person does not wear the mask even after a predetermined allowable time or more already elapses from a time at which the target person included in the thermal image TMP finishes eating, the information output unit 216d outputs an information for notifying that the target person does not wear the mask. For example, the information output unit 216d may output an information for notifying the target person that the target person does not wear the mask to an apparatus that is configured to present an information to the target person (for example, a display that is placed near the target person). As a result, the target person can understand to wear the mask. For example, the information output unit 216d may output an information for notifying a worker of the restaurant at which the target person stays that the target person does not wear the mask to an apparatus that is configured to present an information to the worker (for example, a mobile terminal of the worker). As a result, the worker can ask the target person to wear the mask.

As described above, in the fourth example embodiment, the information processing apparatus 2d is capable of directly or indirectly encouraging the target person that does not wear the mask in the restaurant to wear the mask.

### (5) Supplementary Note

With respect to the example embodiments described above, the following Supplementary Notes will be further disclosed.

[Supplementary Note 1]

An information processing apparatus includes:
an obtaining unit that obtains an infection information relating to an infected person that catches an infection disease; and
an identifying unit that identifies, as a first contact person that possibly contacts the infected person, at least one target person staying in a stay area in which the infected person stays in at least a part of a time period during which the infected person stays in the stay area from a plurality of target persons based on the infection information and a history information that is generated based on a plurality of target person images that indicate the plurality of target persons, respectively.

[Supplementary Note 2]

The information processing apparatus according to Supplementary Note 1, wherein
the identifying unit identifies, as a second contact person that is possibly affected by the infection disease, at least one target person staying in the stay area in at least a part of a time period before a predetermined first time elapses from a time at which the infected person leaves the stay area from the plurality of target persons based on the infection information and the history information.

[Supplementary Note 3]

The information processing apparatus according to Supplementary Note 2, wherein
the identifying unit sets a contact level of the first contact person to the infected person to be a first level,
the identifying unit sets a contact level of the second contact person to the infected person to be a second level that indicates that a degree of a contact is lower than that of the first level.

[Supplementary Note 4]

The information processing apparatus according to Supplementary Note 2 or 3, wherein
when a plurality of second contact persons staying in the stay area in at least partially different time periods, respectively, are identified, the identifying unit (i) sets a contact level of one second contact person, which stays in the stay area before a predetermined second time elapses from a time at which the infected person leaves the stay area, of the plurality of second contact persons to the infected person to be a third level, and (ii) sets a contact level of another second contact person, which stays in the stay area after the second time elapses from the time at which the infected person leaves the stay area, of the plurality of second contact persons to the infected person to be a fourth level that indicates that a degree of a contact is lower than that of the third level.

[Supplementary Note 5]

The information processing apparatus according to any one of Supplementary Notes 1 to 4, wherein
the identifying unit identifies, as a third contact person that is possibly affected by the infection disease, at least one target person staying in a near area that is near the stay area in at least a part of the time period during which the infected person stays in the stay area from the plurality of target persons based on the infection information and the history information.

[Supplementary Note 6]

The information processing apparatus according to Supplementary Note 5, wherein
the identifying unit sets a contact level of the first contact person to the infected person to be a first level,
the identifying unit sets a contact level of the third contact person to the infected person to be a fifth level that indicates that a degree of a contact is lower than that of the first level.

[Supplementary Note 7]

The information processing apparatus according to any one of Supplementary Notes 1 to 6, wherein
when a measured target person a body temperature of which is already measured exists in the plurality of target persons, the history information further includes an information relating to a measured result of the body temperature of the measured target person and a time at which the body temperature of the measured target person is measured,
the information processing apparatus further includes a permitting unit that (i) determines based on the history information whether or not a condition that the body temperature of the measured target person is already measured within a given time from a time at which the measured target person wants to enter a restriction area and the measured body temperature is lower than an allowable threshold value is satisfied, and (ii) permits the measured target person to enter the restriction area when the condition is satisfied even when the body temperature of the measured target person is not measured at a timing of an entry to the restriction area, when the measured target person wants to enter the restriction area to which the entry is not permitted unless the body temperature of the measured target person is measured.

[Supplementary Note 8]

The information processing apparatus according to any one of Supplementary Notes 1 to 7, wherein
the obtaining unit obtains a thermal image from a thermal camera that is configured to generate the thermal image indicating a body temperature of a captured person by capturing an image of the captured person,
the information processing apparatus further includes a determining unit that determines that the captured person impersonates to be another person when a periodical fluctuation of the body temperature is not observed in at least one of a first part, which is covered by a mask, and a second part, which is adjacent to an outer edge of the mask, of a face of the captured person indicated by the thermal image.

[Supplementary Note 9]

The information processing apparatus according to any one of Supplementary Notes 1 to 8, wherein
the obtaining unit obtains a thermal image from a thermal camera that is configured to generate the thermal image indicating a body temperature of a customer by capturing an image of the customer staying in a restaurant,
the information processing apparatus further includes an outputting unit that determines based on the thermal image whether or not the customer wears a mask and outputs an information for notifying that the customer does not wear the mask when it is determined that the customer does not wear the mask even after a predetermined allowable time or more elapses from a time at which the customer finishes eating.

[Supplementary Note 10]

An information processing method including:
obtaining an infection information relating to an infected person that catches an infection disease; and
identifying, as a first contact person that possibly contacts the infected person, at least one target person staying in a stay area in which the infected person stays in at least a part of a time period during which the infected person stays in the stay area from a plurality of target persons based on the infection information and a history information that is generated based on a plurality of target person images that indicate the plurality of target persons, respectively.

[Supplementary Note 11]

A recording medium on which a computer program that allows a computer to execute an information processing method is recorded,
the information processing method including:
obtaining an infection information relating to an infected person that catches an infection disease; and
identifying, as a first contact person that possibly contacts the infected person, at least one target person staying in a stay area in which the infected person stays in at least a part of a time period during which the infected person stays in the stay area from a plurality of target persons based on the infection information and a history information that is generated based on a plurality of target person images that indicate the plurality of target persons, respectively.

[Supplementary Note 12]

A computer program that allows a computer to execute an information processing method,
the information processing method including:
obtaining an infection information relating to an infected person that catches an infection disease; and
identifying, as a first contact person that possibly contacts the infected person, at least one target person staying in a stay area in which the infected person stays in at least a part of a time period during which the infected person stays in the stay area from a plurality of target persons based on the infection information and a history information that is generated based on a plurality of target person images that indicate the plurality of target persons, respectively.

At least a part of the feature of each embodiment described above may be combined with at least other part of the feature of each embodiment described above. A part of the feature of each embodiment described above may not be used. Moreover, the disclosures of all documents (for example, publications) that are cited in the present disclosure described above are incorporated in the present disclosure by reference if it is legally permitted.

The present disclosure is allowed to be changed, if desired, without departing from the essence or spirit of the invention which can be read from the claims and the entire specification, and an information processing apparatus, an information processing method and a recording medium, which involve such changes, are also intended to be within the technical scope of the present disclosure.

### DESCRIPTION OF REFERENCE CODES

1 camera
1b thermal camera
2 information processing apparatus
21 arithmetic apparatus
211 information obtaining unit
212 authentication unit
213 contact person identification unit
214b entry management unit
215c impersonation determination unit
216d information output unit
3 communication network
SYS information processing system

What is claimed is:

1. An information processing apparatus comprising:
at least one memory configured to store instructions; and
at least one processor configured to execute the instructions to:
obtain an infection information relating to an infected person that catches an infection disease; and
identify, as a first contact person that possibly contacts the infected person, at least one target person that stays in a stay area in which the infected person stays in at least a part of a time period during which the infected person stays in the stay area from a plurality of target persons based on the infection information and a history information that is generated based on a plurality of target person images that indicate the plurality of target persons, respectively.

2. The information processing apparatus according to claim 1, wherein
the at least one processor is configured to execute the instructions to identify, as a second contact person that is possibly affected by the infection disease, at least one target person that stays in that stays area in at least a part of a time period before a predetermined first time elapses from a time at which the infected person leaves the stay area from the plurality of target persons based on the infection information and the history information.

3. The information processing apparatus according to claim 2, wherein
the at least one processor is configured to execute the instructions to:
set a contact level of the first contact person to the infected person to be a first level; and
set a contact level of the second contact person to the infected person to be a second level that indicates that a degree of a contact is lower than that of the first level.

4. The information processing apparatus according to claim 2, wherein
when a plurality of second contact persons that stays in the stay area in at least partially different time periods, respectively, are identified, the at least one processor is configured to execute the instructions to (i) set a contact level of one second contact person, which stays in the stay area before a predetermined second time elapses from a time at which the infected person leaves the stay area, of the plurality of second contact persons to the infected person to be a third level, and (ii) set a contact level of another second contact person, which stays in the stay area after the second time elapses from the time at which the infected person leaves the stay area, of the plurality of second contact persons to the infected person to be a fourth level that indicates that a degree of a contact is lower than that of the third level.

5. The information processing apparatus according to claim 1, wherein
the at least one processor is configured to execute the instructions to identify, as a third contact person that is possibly affected by the infection disease, at least one target person that stays in a near area that is near the stay area in at least a part of the time period during which the infected person stays in the stay area from the plurality of target persons based on the infection information and the history information.

6. The information processing apparatus according to claim 5, wherein
the at least one processor is configured to execute the instructions to:
set a contact level of the first contact person to the infected person to be a first level; and
set a contact level of the third contact person to the infected person to be a fifth level that indicates that a degree of a contact is lower than that of the first level.

7. The information processing apparatus according to claim 1, wherein
when a measured target person a body temperature of which is already measured exists in the plurality of target persons, the history information further includes an information relating to a measured result of the body temperature of the measured target person and a time at which the body temperature of the measured target person is measured,
the at least one processor is further configured to execute the instructions to (i) determine based on the history information whether or not a condition that the body temperature of the measured target person is already measured within a given time from a time at which the measured target person wants to enter a restriction area and the measured body temperature is lower than an allowable threshold value is satisfied, and (ii) permits the measured target person to enter the restriction area when the condition is satisfied even when the body temperature of the measured target person is not measured at a timing of an entry to the restriction area, when the measured target person wants to enter the restriction area to which the entry is not permitted unless the body temperature of the measured target person is measured.

8. The information processing apparatus according to claim 1, wherein
the at least one processor is configured to execute the instructions to:
obtain a thermal image from a thermal camera that is configured to generate the thermal image indicating a body temperature of a captured person by capturing an image of the captured person; and
determine that the captured person impersonates to be another person when a periodical fluctuation of the body temperature is not observed in at least one of a first part, which is covered by a mask, and a second part, which is adjacent to an outer edge of the mask, of a face of the captured person indicated by the thermal image.

9. The information processing apparatus according to claim 1, wherein
the at least one processor is configured to execute the instructions to:
obtain a thermal image from a thermal camera that is configured to generate the thermal image indicating a body temperature of a customer by capturing an image of the customer that stays in a restaurant; and
determine based on the thermal image whether or not the customer wears a mask and output an information for notifying that the customer does not wear the mask when it is determined that the customer does not wear the mask even after a predetermined allowable time or more elapses from a time at which the customer finishes eating.

10. An information processing method comprising:
obtaining an infection information relating to an infected person that catches an infection disease; and
identifying, as a first contact person that possibly contacts the infected person, at least one target person that stays in a stay area in which the infected person stays in at least a part of a time period during which the infected person stays in the stay area from a plurality of target persons based on the infection information and a history information that is generated based on a plurality of target person images that indicate the plurality of target persons, respectively.

11. The non-transitory recording medium on which a computer program that allows a computer to execute an information processing method is recorded,
the information processing method comprising:
obtaining an infection information relating to an infected person that catches an infection disease; and
identifying, as a first contact person that possibly contacts the infected person, at least one target person that stays in a stay area in which the infected person stays in at least a part of a time period during which the infected person stays in the stay area from a plurality of target persons based on the infection information and a history information that is generated based on a plurality of target person images that indicate the plurality of target persons, respectively.
